(19) United States
(12) Patent Application Publication
(10) Pub. No.: US 2003/0104613 A1
(43) Pub. Date: Jun. 5, 2003

(54) Method for producing adherent animal cells

(76) Inventor: Catherine Gerdil, Tassin (FR)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/148,514

(22) PCT Filed: Dec. 4, 2000
(86) PCT No.: PCT/FR00/03377

(30) Foreign Application Priority Data
Dec. 3, 1999 (FR) 99/15303

(51) Int. Cl. C12N 5/02; C12N 5/08

(52) U.S. Cl. 435/325; 435/366; 435/404

(57) ABSTRACT

The invention concerns a culture medium containing a polyvinylpyrrolidone as substitute for the serum of animal origin for producing animal or human adherent cells under stirring by increasing the cell proliferation or by decreasing cell death rate. Said medium is also suitable for producing viruses.

The present invention relates to a method for producing adherent animal cells in a culture medium free of serum of animal origin, for the manufacture of viral vaccines, and also to the use of a polyvinylpyrrolidone (PVP) molecule.

### PRIOR ART

Culture media generally consist of a base medium, which contains the nutrient elements that are essential for the growth of the cells, supplemented with blood serum, usually donor calf serum or fetal calf serum.

Culture media free of serum of animal origin have been reported to induce the proliferation of nonadherent cells in suspension, especially the hybridoma cell line culture (Kovar and Franek, Biotechnol. Lett. 9:259-264 (1987); Murakami H., in Growth of cells hormonally defined media (G. Sato et al., eds), Cold Spring Harbor Laboratories, Cold Spring Harbor, N.Y., p. 711-715 (1982); patent JP 63084487).

It is known that culture media free of serum of animal origin are unsuitable for culturing adherent cells (Kovar J., In vitro Cell. Dev. Biol. 25:395-396 (1989)). The attachment of adherent cells to a support requires attachment factors provided by the culture medium, such as fibronectin, vitronectin, laminin and collagen (Koller M. R. and Papoutsakis E. T., Bioprocess. Technol. 20:61-110 (1995). These attachment factors are present essentially in serum of animal origin (Hayman E., et al., Exp. Cell. Res. Vol. 160:245 (1985)).

Patent application WO 88/00967 describes a process for culturing adherent mammalian cells in a biogenerator, comprising a step of culturing in a medium containing serum to promote the attachment of the cells to a support and to allow the proliferation of the cells up to a desired concentration, followed by a step in which the culture medium is eliminated and then replaced with a serum-free medium, moreover containing a polyvinylpyrrolidone, polyethylene glycol or a polymer consisting of one or more oxide groups to protect the adherent cells by reducing the film-forming drainage around the cells. This process is used for the production of recombinant molecules by cells. It is not mentioned that a PVP acts on the cell proliferation.

Patent application FR 2 635 008 describes a serum-free defined culture medium, moreover containing a PVP with a molecular weight of 40 kd (PVP 40) at a concentration of at least 3% in the medium, to promote the production of interleukin-2 in the culture supernatant by a recombinant adherent CHO line cultured on microcarriers placed in a reactor subjected to mechanical agitation. It is also clearly indicated that a culture medium with a PVP 40 is not better than the same medium without PVP 40 for ensuring the growth of this line (page 7, lines 15 to 25; page 8, lines 5 to 8).

Patent application WO 98/24883 describes the composition of a serum-free culture medium containing a steroidal compound and, secondarily, Pluronic F68 as surfactant and a 10-kd PVP (PVP 10) as detoxifying agent, to promote the growth of adherent lines, such as the Vero line placed in a static environment, that is to say in an environment in which the line is not subjected to agitation flow of the culture medium. The presence of sterols in the culture medium is essential to observe cell growth.

According to the teaching of the prior art, it may reasonably be doubted that a serum-free medium containing a PVP can ensure the multiplication of adherent lines when they are placed in a uniformly agitated culture medium, this being an environment that is often encountered during the production of cells in large amounts or on a large scale. Specifically, patent application FR 2 635 008 shows that a PVP used as a serum substitute does not increase the multiplication of cells adhering to microcarriers subjected to a uniform ambient agitation. The article by Gyun M. et al. (Hybridoma, Vol 8: 639-645) moreover points out that serum is essential when the culture medium is regularly agitated (page 639).

There is a need in the field of cell culturing to find a substitute for serum of animal origin, since its use is very restrictive. As a product of biological origin, serum is subject to very strict regulatory constraints for use in order to avoid the transmission of animal viruses to man. Furthermore, the components of serum are not all very well identified and vary in amount from one batch to another, whence arises the need to control the biological activity of these batches and more particularly their action on cell growth so as to select only the best. The presence of a large amount of proteins in the serum may also singularly complicate the methods for isolating and purifying the cell components and cell derivatives such as viruses.

There is also a need to find a serum-free culture medium which promotes the growth of cells in a regularly agitated medium, which is an environment that is generally encountered during the production of cells in large amounts. Mechanical agitation or agitation mediated by a flow of gas is in fact a necessary condition since it promotes the supply of nutrients, but it also has the drawback of making the cells fragile on account of the shear forces it generates.

The subject of the present invention overcomes these needs by proposing a novel method for producing a particular class of animal or human cells.

### SUMMARY OF THE INVENTION

To this end, the present invention relates firstly to the use of a polyvinylpyrrolidone with an average molecular weight of between 20 kd and 360 kd in a culture medium free of serum of animal origin, to increase the proliferation of adherent nonrecombinant animal or human cells subjected to an agitation flow of the medium.

The invention relates in particular to the use of a polyvinylpyrrolidone with an average molecular weight of between 20 kd and 360 kd in a culture medium free of serum of animal origin, in which the proliferation index of the cells in this medium is greater than 1.5.

The invention relates to the use of a polyvinylpyrrolidone with an average molecular weight of between 20 kd and 360 kd in a culture medium free of serum of animal origin, to promote the adhesion of adherent animal or human cells subjected to an agitation flow of the medium.

The invention also relates to the use of a polyvinylpyrrolidone with an average molecular weight of between 20 kd and 360 kd in a culture medium free of serum of animal origin, to reduce the death rate of adherent animal or human cells subjected to an agitation flow of the medium.

The invention also relates particularly to the use of a noncrosslinked polyvinylpyrrolidone.

The polyvinylpyrrolidone preferably has an average molecular weight of 40 kd.

According to another aspect, the invention concerns the percentage of polyvinylpyrrolidone in the culture medium, which is between 0.01% and 2%.

Preferably, the percentage of polyvinylpyrrolidone in the culture medium is 0.1%.

The invention also relates to the use of a polyvinylpyrrolidone in a culture medium that has a chemically defined composition.

In another aspect, the invention also relates to the use of a polyvinylpyrrolidone for the production of nonrecombinant adherent human or animal cells in batch or feed batch mode or in continuous culture mode.

The invention also relates to a process for producing adherent nonrecombinant animal or human cells, comprising:

(i) a first step in which a suspension of cells in a serum-free culture medium containing a polyvinylpyrrolidone with an average molecular weight of between 20 kd and 360 kd is inoculated in a culturing device comprising an adhesion support;

(ii) a second step in which the cells multiply under agitation in the same culture medium;

(iii) a third step in which the cells are harvested when they have reached a growth stage.

In one embodiment of the process according to the invention, the polyvinylpyrrolidone has an average molecular weight of 40 kd.

In another embodiment of the process according to the invention, the percentage of polyvinylpyrrolidone in the culture medium is between 0.01% and 2%.

In a preferred embodiment, the percentage of polyvinylpyrrolidone in the culture medium is 0.1%.

In another embodiment of the process according to the invention, the adhesion support consists of microcarriers.

In another particular embodiment of the process according to the invention, the microcarriers consist of a dextran matrix (cytodex 1).

In another particular embodiment, the culture device is a biogenerator.

In one aspect of the process according to the invention, the culture mode is the batch or feed batch mode or the continuous culture mode.

Finally, in one particular aspect of the process for producing cells according to the invention, the cells produced are cells of the Vero or MRC5 line.

Finally, a subject of the invention is a process for producing viruses, according to which:

(i) cells are infected with viruses in a first culture medium;

(ii) the viruses are left to propagate and are harvested in a second culture medium;

at least one of the two media being a medium free of serum of animal origin and containing a polyvinylpyrrolidone with an average molecular weight of beween 20 kd and 360 kd.

In one embodiment of the process for producing viruses according to the invention, the first culture medium is free of serum of animal origin and contains a polyvinylpyrrolidone with an average molecular weight of between 20 kd and 360 kd.

In another embodiment of the process for producing viruses according to the invention, the second culture medium is free of serum of animal origin and contains a polyvinylpyrrolidone with an average molecular weight of between 20 kd and 360 kd.

In one particular embodiment of the process for producing viruses according to the invention, the viruses are produced from cells of the Vero or MRC5 line.

In an even more particular embodiment of the process for producing viruses according to the invention, the viruses are polio virus, rabies virus, hepatitis A virus or rubella virus.

Contrary to all expectation, a PVP of from 20 kd to 360 kd, and preferably a PVP of 40 kd, in a serum-free culture medium can ensure, with an efficacy comparable to that of a medium containing serum, the growth of adherent cells subjected to an agitation flow of the medium. More surprisingly, a 10-kd PVP tested on the same cells and used under the same conditions has no significant effect on the growth, whereas patent application WO 98/24883 concerns a defined culture medium containing a 10-kd PVP and steroidal compounds in the presence of surfactants to promote the growth of adherent lines placed in a static environment.

### DETAILED DESCRIPTION OF THE INVENTION

In the context of the present invention, various terms used are defined below:

“Polyvinylpyrrolidone” or “PVP” means a sequence of the 1-vinyl-2-pyrrolidone monomer via covalent bonding, so as to obtain a polymer of the monomer. When the sequence is linear and contains no branching, it is considered that the polyvinylpyrrolidone is noncrosslinked.

“Culture medium having a chemically defined composition” means a medium comprising a defined number of components that are molecularly well characterized and used at concentrations that are also well determined. A culture medium containing serum of animal origin is thus not a chemically defined culture medium.

“Culture medium free of serum of animal origin” means a culture medium containing no serum or product extracted from sera of animals and especially those originating from mammals, birds, fish or crustaceans. As regards the culture medium, it may have a chemically defined composition or a composition that is not chemically defined if it contains, for example, extracts of microorganisms, of yeasts or of fungi or even of plants that are not chemically well characterized.

“Adherent cells” means cells established in lines or cells derived directly from the extraction of healthy or tumorous animal or human tissues that have need of a solid support to multiply and grow normally. An essential characteristic of adherent cells is that they form a uniform unicellular layer on their support due to the phenomenon of contact inhibition. Cells that have no need of a solid support to multiply are consequently excluded. In general, these cells grow in suspension in the culture medium, such as, for example, hybridoma lines. The adherent lines can be derived from primary cultures of healthy or tumoral cells, but may also be obtained by transforming cells using immortalizing agents. The adherent lines used may be mortal (limited lifetime) or immortal (unlimited lifetime).

“Agitation flux of the medium” means a culture medium that is agitated mechanically and/or under the action of a stream of gas.

“Proliferation index” means the ratio between the maximum number of cells obtained per ml of culture medium containing a PVP as serum substitute and the maximum number of cells obtained per ml of the same culture medium but lacking PVP. To calculate this index, it is understood that the cells and the culture conditions are identical. The maximum number of cells obtained in each of the two media tested is taken into account only when the growth curve observed for each of the media has reached a “stationary” or “steady” phase, indicating that the cell proliferation has stopped. A proliferation index≧1+2σ (σ being the uncertainty regarding the cell counting, expressed in %) means that the medium containing a PVP increases the cell proliferation.

“Batch mode” means a mode of culturing in a closed medium without supplying additional nutritional elements, and without a device for removing the toxic waste which accumulates during the culturing.

“Fed-batch mode” means a mode of culturing in a semi-closed medium with at least a limited supply of additional nutritional elements during the culturing, but this mode of culturing does not comprise a device for removing the toxic waste that has accumulated.

“Continuous culturing mode” means a mode of culturing in which fresh culture medium is constantly supplied to replace at the same time an equivalent amount of spent medium which is removed.

“Continuous culturing mode under perfusion” means a continuous culturing mode in which there is an additional device that retains the cells.

“Adhesion support” means a solid surface whose outer chemical components in contact with the culture medium allow the attachment of the adherent cells.

“Bio-generator” means a culture tank, generally made of stainless steel, with a volume greater than 2 liters, comprising an agitation system, a device for injecting a stream of CO₂ gas and an oxygenation device. It is equipped with probes measuring the internal parameters of the biogenerator, such as the pH, the dissolved oxygen, the temperature, the tank pressure or certain physicochemical parameters of the culture (for instance the consumption of glucose or of glutamine or the production of lactates and ammonium ions). The pH, oxygen and temperature probes are connected to a bioprocessor which permanently regulates these parameters.

“Microcarriers” means porous or nonporous spherical microbeads 100 to 200 μm in diameter, the density of which is very slightly greater than that of the culture medium, which are coated with an adhesive electrostatic matrix. The microcarriers are maintained in suspension by mechanical agitation.

“Infection step” means the contact time required for infectious agents with a strict intracellular reproductive cycle such as viruses, prepared in the form of a suspension in a suitable medium, termed the “infection medium” to be able to enter the cell.

“Propagation step” means the time required for infectious agents with a strict intracellular reproductive cycle, such as viruses, to be able to effect their complete reproductive cycle in the cell and for them to be produced in a mature form. The propagation step takes place in a suitable culture medium, termed the “propagation medium”, which may be different from the infection medium. The mature forms of certain viruses, such as chicken pox virus, remain intracellular; in this case, the virus is harvested by means of lysing the infected cells. In other cases, the mature forms are extracellular and can reinfect other cells that remained unharmed by reinitializing new intracellular reproductive cycles. The virus may be harvested in a single operation by withdrawal of the propagation medium, or at regular intervals, the withdrawn propagation medium being replaced with fresh medium, up to the point of depletion of the intracellular reproductive cycle by complete destruction of the cellular layer.

The invention thus relates to the use of a polyvinylpyrrolidone, that is preferably noncrosslinked, with an average molecular weight of between 20 kd and 360 kd (PVP), and preferably a PVP with an average molecular weight of 40 kd, in a culture medium free of serum of animal origin, to promote the multiplication of adherent animal or human cells subjected to an agitation flux of the medium. The optimum effect on the proliferation of the adherent cells such as, for example, on chick embryo cells, on mouse lines such as the line 3T3, NTCT, WEHI, on hamster lines such as the line BHK or CHO, on canine lines such as the line MDCK or dog kidney primary cells, on porcine lines such as the line PK15, on simian lines such as the line Vero, LLC-MK2, FRHL2, or on human lines such as the line MRC5, HeLa, ECV or A431 or melanoma lines such as the line A375 is obtained when a PVP is used at a concentration in the culture medium of between 0.01% and 2% (weight/volume). A person skilled in the art is capable of adapting the working concentrations of the PVP in the culture medium as a function of the average molecular weight of said PVP. Specifically, it is known that the higher the average molecular weight of the PVP, the higher the viscosity of a medium containing it. Too high a viscosity is an inconvenience for cell growth. As a guide, a person skilled in the art may refer to the viscosity value of a medium containing a 40-kd PVP at a concentration of 0.1%, which is compatible with very good cell growth, to determine the working concentrations of a PVP in the average molecular weight range recommended to perform the invention.

A PVP, with an average molecular weight of between 20 kd and 360 kd and preferably of 40 kd, used at a concentration of between 0.01% and 2% in a culture medium free of serum of animal origin also promotes the adhesion of these cells or of these cell lines to their support in the hours following their inoculation but despite an agitation of the culture medium.

Finally, it is noted that a PVP used under the same conditions in a culture medium free of serum of animal origin substantially reduces tbe cell death rate throughout the culture period.

A PVP according to the invention is generally characterized by its average molecular weight, but may also be defined by means of its K value, which takes account not only of the average molecular weight of a PVP, but also of the variations in molecular weight on either side of the average value. To calculate the K value, reference is made to the equation as defined in the article Cryobiology, 8, 453-464 (1971): the K value is calculated from the relative viscosity of a 1% PVP solution according to the formula

Log η rel/C=75K₀²/(1+1.5 K₀C)+K₀

K=1000 K₀

C represents the concentration in grams of PVP per 100 ml of medium,

η rel is the viscosity of the solution compared to that of the solvent.

This K value definitively represents the intrinsic viscosity of a PVP which, for all intents and purposes of the invention, should have a value of between 20 and 100 for a PVP with an average molecular weight of between 20 kd and 360 kd.

The base culture medium into which is incorporated a PVP according to the invention may be MEM, MEM-α, DMEM, RPMI, ISCOVE, Ham F12, HAM F10, M199, L15, 6M, or NCTC109 medium, Fischer medium, Waymouth medium, Nephros medium (defined medium produced by Biowhittaker under the reference number 12-735Q), VPSFM medium (defined medium supplied by the company Gibco Life Technologies and having the reference number 11002086), Williams medium or mixtures of these base media. These base media may be enriched according to the needs of the cells, with additional nutrient factors such as, for example, sugars such as glucose, amino acids such as glutamine, a cocktail of nonessential amino acids (supplied especially by the company Gibco Life Technologies) or of essential amino acids or of peptides and more particularly of peptides of plant origin, acids or acid salts such as sodium pyruvate, EDTA salts, citric acid derivatives or more generally derivatives of acids involved in the Krebs cycle, alcohols such as ethanol, amino alcohols such as ethanolamine, vitamins such as vitamin C and vitamin E, antioxidants such as glutathione or selenium, fatty acids with saturated or unsaturated chains such as linoleic acid, arachidonic acid, oleic acid, stearic acid or palmitic acid, lipids or lipopeptides, and also with phospholipids such as lecithins and preferably lecithins of plant origin or with precursors thereof. The addition of a buffer solution based on HEPES or bicarbonates may prove to be necessary for certain fragile cell cultures or for cultures producing large amounts of CO₂, or optionally to buffer culture media that are highly supplemented with acids. In general, care will be taken to ensure that the pH of the culture medium remains between 6 and 8, usually between 7 and 8 and more specifically between 7.2 and 7.5. As far as is possible, care will be taken to ensure that the culture medium remains isotonic.

According to the particular needs of certain types of cells, the subject of the invention may advantageously be exploited by adding to the culture medium steroidal derivatives such as cortisol, growth factors of synthetic or recombinant origin such as, for example, cytokines, growth hormone, IGFs (insulin growth factors), EGF (epidermal growth factor), FGF (fibroblast growth factor), PDGF (platelet-derived growth factor), and attachment factors such as recombinant collagen. However, these factors are not absolutely necessary to perform the invention. Even though a PVP is preferentially used in a chemically defined culture medium, certain cell cultures, especially primary cultures obtained from organ explants, may require culture supplements based on bacterial, yeast or even plant extracts.

It is clearly understood that the list of additives is merely indicative and should not in any way be considered as restrictive for a person skilled in the art wishing to reproduce the subject of the invention.

Another aspect of the invention concerns various culturing processes that may be used to ensure the growth of adherent cells with the aid of a culture medium in accordance with the invention. The culturing of adherent cells using a culture medium according to the invention may be performed in “batch mode”, “feed batch mode” or “continuous culturing mode” when the cell growth reaches its steady state in an interval of 7 days and when the maximum cell concentration does not exceed 3×10⁶ cells/mL. On the other hand, when it is desired to obtain maximum cell concentrations of greater than 3×10⁶ cells/mL and/or when the culturing time is longer, the initial culture medium according to the invention becomes too depleted in certain essential nutrients such as glucose or glutamine, while the toxic wastes derived from the cell metabolism, for instance lactates or ammonia, accumulate. In this case, the “fed-batch” mode or the “continuous culturing” mode is used. The “fed-batch” mode consists in adding from time to time some of the base nutrient components contained in the initial culture medium according to the invention which have been consumed too quickly by the cells. This generally involves adding glutamine, or cocktails of amino acids, glucose and occasionally, depending on the cultures, lipid compounds. In the “continuous culturing” mode, the problem of accumulation of the waste is also overcome due to the fact that the culture medium according to the invention is constantly renewed. A person skilled in the art, by following the change in the parameters of the cell metabolism, for instance the measurement of the glucose or glutamine consumption, or by following the levels of ammonia and lactates in the culture medium, is entirely capable of determining the culture mode that is best suited. The culturing devices that allow agitation of the culture medium comprise rolling flasks, flasks of “spinner” type, and biogenerators. The culture supports may consist of the walls of the flasks themselves without necessarily being treated beforehand with particular adhesive agents. Microcarriers which considerably increase the surface area available to the adherent cells without, however, significantly increasing the required volume of culture medium are also used. Nevertheless, this type of advantageous support requires permanent agitation and often good oxygenation of the culture medium. The use of microcarriers in rolling flasks, flasks of spinner type or biogenerators may be exploited. Depending on the cell types used, the microcarriers may consist of a dextran matrix substituted with N,N-diethylaminoethyl groups (Cytodex 1 and Cytodex 2), or optionally with attachment factors of synthetic or recombinant origin, for instance poly-D-lysine, recombinant collagen or the RGD peptide consisting of the sequence of arginine, glycine and aspartic acid. The microcarriers may also be made of polystyrene, glass, cellulose or polyacrylamide. They may be used at a concentration of between 1 g/liter and 100 g/liter of culture medium according to the invention. Microcarriers of the Cytodex 1 type are preferably used at a concentration of between 3 g/liter and 30 g/liter and even more preferably at a concentration of between 3 g/liter and 15 g/liter.

When the microcarriers of the Cytodex 1 type are placed in a biogenerator at a concentration of 3 g/liter, the maximum cell concentration that may be obtained with a medium according to the invention depends on the size of the cells and the initial cell inoculation conditions. Specifically, in order for there to be uniform coverage of the surface of the microcarriers with the cells, it is necessary for each microcarrier to be colonized by at least one cell during the inoculation. It has been found that this condition is satisfied when the initial cell inoculation concentration is between 15 and 50×10³ cells per cm² of microcarrier surface area. However, under these inoculation conditions, the maximum cell concentration does not generally exceed 3×10⁶ cells/mL in the biogenerator, when large cells are cultured, such as the cells of the Vero or MRC5 line. The growth threshold is generally obtained within a period of 7 days after initiation of the culture, such that the “batch”, “feed batch” or “continuous culturing” mode may be used. On the other hand, when the microcarriers of Cytodex 1 type are used at a concentration of 15 g/liter, cell densities of Vero or MRC5 cells exceeding 5×10⁶ cells/mL are obtained, under the same optimum inoculation conditions, using a medium according to the invention. The “feed batch” mode or the “continuous culturing” mode is then used to overcome the deficit of essential nutrients and/or the problem of the accumulation of toxic waste. Whatever the mode used, the culture medium is permanently agitated, for example using a mechanical agitation paddle of the “delta” type (patent Fr 80/18608). When a mode of “continuous culturing under perfusion” is used, the agitation paddle is preferably placed facing the device which retains the cells in the biogenerator, this device being, for example, a strainer. Whatever the culture mode used, the medium is also regularly oxygenated by means of a hollow dip tube fed with oxygen. It is also possible to use a process of oxygenation of the medium according to the invention outside the biogenerator, also known as an oxygenator. The oxygenator may consist of a system of hollow fibers whose oxygen-permeable polysulfone membrane allows the transfer of oxygen into the culture medium. The culture medium of the biogenerator is also fed with CO₂ when the pH falls. The agitation speed of the medium, and also the oxygen content of the medium according to the invention are parameters that are readily controlled by a person skilled in the art. The proliferation index obtained using a culturing process according to the invention and especially the process of culturing in a biogenerator with cytodex-based microcarriers, often exceed 1.5 and are occasionally close to those observed with culture media containing serum of animal origin used for the industrial production of cells.

A final aspect of the invention concerns a process for producing viruses using a medium according to the invention. This process is composed of two phases. In a first phase, the functions of the PVP are exploited on the adhesion, the growth and the beneficial effect on the viability of the cells to produce the virus-sensitive cells using one of the culturing processes recommended according to the invention. Homogeneous cell population in which most of the cells have conserved their metabolic integrity, which is the corollary of good cell viability, is obtained. As a result, the cells are in a state of maximum receptivity to the virus and furthermore are in an optimum arrangement to actively produce the virus. It has also been noted, surprisingly, that the culture medium according to the invention used during the first phase of the process is also suitable as a viral infection medium and/or as a viral propagation medium for the production of viruses, which constitutes the second phase of the process. Specifically, a PVP incorporated into a base culture medium free of serum of animal origin can promote the entry of the virus into the cell and/or the replication of the virus inside said cell. The media that may be used are the same base culture media described for the production of cells to which is added a PVP with an average molecular weight of between 20 kd and 360 kd, but preferably of 40 kd; the final concentration in the medium being between 0.01% and 2%, and preferably 0.1%. These media have especially been used for the production of polio virus and rabies virus from cells of the Vero or MRC5 line, and for the production of rubella virus or hepatitis A virus from cells of the MRC5 line. Other extracellular viruses may also be produced using this process, such as yellow fever virus, measles virus, mumps or flu virus. This process may also be used for the production of intracellular viruses such as chicken pox virus and more generally for the production of infectious agents with a strict intracellular reproductive cycle including parasites such as the agent responsible for malaria or toxoplasmosis. The viral productions obtained according to this process are then conventionally processed to manufacture viral vaccines that may be used in human or veterinary medicine. Thus, by combining the process for producing cells and the process for producing viruses according to the invention, the use of culture media based on serum of animal origin is dispensed with for all the steps of the manufacture of viral vaccines, with virus production yields that are better than those obtained with PVP-free media.

The object of the present invention will now be understood more clearly on reading the examples that follow, which do not, however, have any limiting nature.

### EXAMPLE 1

### Study of the Growth of a Population of Vero Cells Cultured in a Biogenerator Using Chemically Defined Culture Media Containing a PVP

1A—Characteristics of the Vero Line Used

The strain used is derived from the American Type Culture Collection (ATCC) under the reference code ATCC cclVERO F 1415, passage 124. A working cell bank at the 137^{th} passage of this strain was made. The Vero cells are cultured by successive passages using the working bank in culture medium supplemented with calf serum up to the 140^{th} passage. They then undergo 2 to 3 passages in one or other of the two following serum-free defined media: the first consisting of equal parts of Williams medium and Nephros medium, the second consisting of equal parts of VPSFM medium and of Williams medium, these additional passages being performed so as to have available a sufficient amount of cells to inoculate a biogenerator, a spinner or rolling flasks.

1B—Culture Media and Reagents Used

Preparation of the Chemically Defined Base Culture Media

All the solutions prepared are filtered through a 0.22 μm Millex unit (Millipore) to sterilize them.

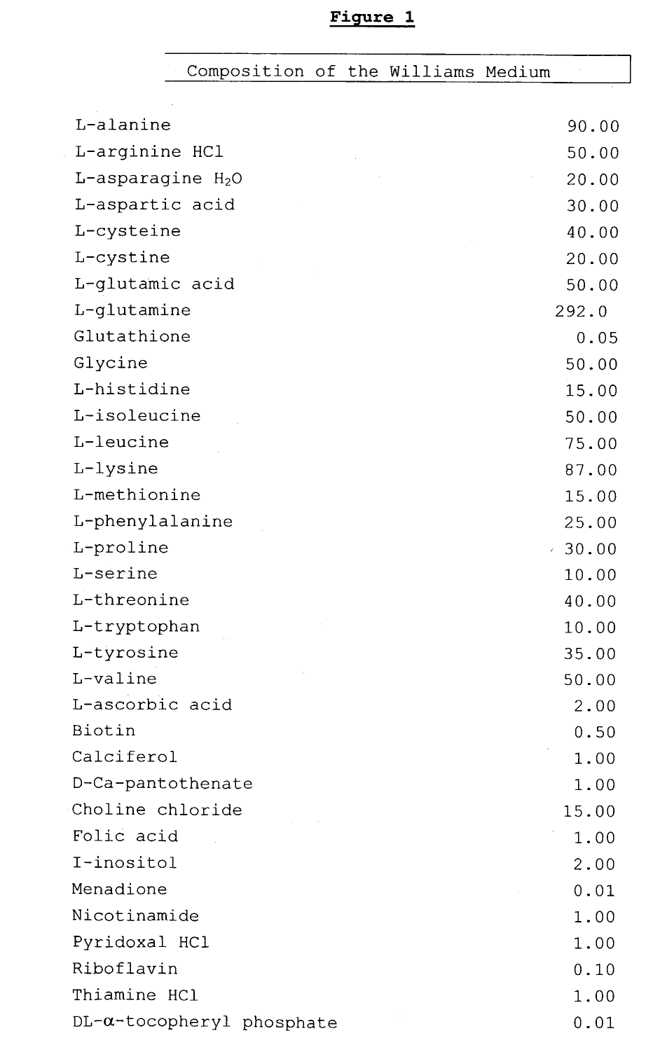

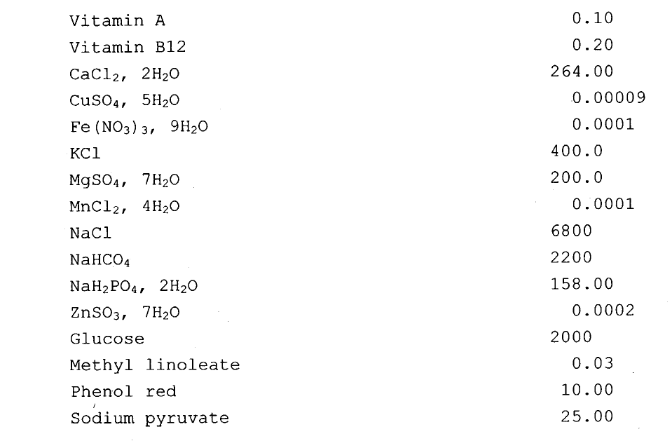

Williams medium (see annexe 1)

| | | |
|---|---|---|
| Mixture of Williams powder (Gibco), | 1 | sachet |
| the components of which, expressed | | |
| in grams, are given in FIG. 1 | | |
| NaHCO₃ (Merck) | 2.20 | g |
| Gentamicin (Unicet) | 50 | mg |

| | |
|---|---|
| ultrafiltered H₂O | qs 11 |

Nephros medium (Biowhittaker—Ref. No. 12-735Q): ready-to-use medium

Extemporaneous addition of:

| | | |
|---|---|---|
| Glutamine 200 mM | 10 | ml/l |
| Neomycin | 1 | ml/l |

VPSFM medium (Gibco Life Technologies—Ref. No. 11002086) ready-to-use

Formulae and Preparations of the Solutions and Buffers Used in Culturing

PBS free of Ca²⁺ and Mg²⁺

| | |
|---|---|
| solution 10 × c (Gibco) | 10 ml |
| ultrafiltered H₂O | qs 11 |

Citrate buffer

| | | |
|---|---|---|
| NaCl | 8.00 | g |
| KCl | 0.20 | g |
| Na₂HPO₄, 2H₂O | 1.25 | g |
| KH₂PO4 | 0.20 | g |
| trisodium citrate, 2H₂O | 7.40 | g |

| | |
|---|---|
| ultrafiltered H₂O | qs 11 |

2.5% Sigma trypsin

| | | |
|---|---|---|
| trypsin recrystallized 2 × (Sigma) | 25.00 | g |
| NaCl (Merck) | 9.00 | g |

| | |
|---|---|
| ultrafiltered H₂O | qs 11 |

The citrate buffer and the Sigma trypsin were filtered through a 0.22 μm Millipack cartridge (Millipore). The PBS was filtered through a 0.22 μm membrane (Sartorius).

Soybean trypsin inhibitor solution at 1 mg per liter

| | | |
|---|---|---|
| Soybean trypsin inhibitor (Boehringer) | 20 | mg |
| Base Williams medium E | 20 | ml |

This solution may be used extemporaneously or stored at −20° C. for 2 to 3 weeks.

1C—Stock Solution of 10% PVP (Weight/Volume)

50 g of 40-kd PVP (Sigma—Ref. 97H0571) or 50 g of 360-kd PVP (Sigma—Ref. P5288) are dissolved in 500 ml of ultrafiltered water followed by a sterilizing filtration through a 0.22 μm Millex unit (Millipore). The various stock solutions thus prepared are stored at +4° C. and added in a suitable amount to the culture medium at the time of use as a function of the desired final concentrations of PVP.

1D—Description of the Biogenerator and of the Spinner

3-Liter Biogenerator.

This consists of a round-bottomed stainless-steel tank equipped with an agitation system consisting of a delta paddle. It is equipped with pH, O₂ pressure and temperature probes and is connected to a BP 2.10 IM control pannel (PMC) which continuously controls the various parameters by means of injecting CO₂, that of the dissolved oxygen content by flushing with air at the surface and a deep injection of oxygen. The temperature is controlled using a Lauda RM6 cryostat which maintains the culture at 37° C. by means of a jacket in which the cryostat water passes in a closed circuit.

Other systems were used for certain continuous culturing tests under perfusion. A perfusion system was installed (Applikon Biosep ADI 1015) involving an ultrasound resonance chamber for removing spent medium while at the same time releasing the microcarriers in the biogenerator. This system requires the use of two pumps, a recycling pump (Masterflex console drive, model 7518-02) and a Minipulse 2 two-head pump (Gilson) for the feeding and discharging of the biogenerator. The whole is managed by an Applikon Biosep ADI 1015 unit controlling the power and frequency of the system and regulating the recycling pump and the resonance chamber.

250 ml Spinner (Integra Biosciences)

These are made of glass with two stoppers at each end. The agitation system requires a magnetic stirrer. The system in the spinner consists of two smooth round rods containing a magnetic bar. Rotation is provided by a Cellspin magnetic plate (Integra Biosciences). The parameters are measured manually.

pH Meter

The pH of the media after preparation and during culturing is monitored using an electronic Minissis 5000 Tacussel pH meter (PHM 220).

1E—Culturing of the Vero Cells in a 3-Liter Biogenerator (or in a 250 ml Spinner) on Microcarriers of the Cytodex 1 Type

Cytodex 1 beads (Pharmacia) are hydrated for 24 hours at a rate of 1 g of beads per 50 ml approximately of phosphate buffer (0.1M, pH=7.4) followed by washing 5 to 10 times in the same buffer until the pH of the buffer turns to normal. The beads are then sterilized for one hour at 121° C. under 1.5-2 bar. Just before use, the microcarriers are rinsed twice with the medium to be used for the cell culturing before being placed at a rate of 3 g/liter of medium in a spinner or a biogenerator in order to produce a low concentration of cells (“low-density culture”) or at a rate of 15 g/liter of medium in a biogenerator in order to produce a high concentration of cells (“high-density culture”). The ready-to-use Vero cells are inoculated at a rate of 30×10³ cells per cm² of culturable surface area. The final volume of medium corresponds to the volume recommended for the culture system used (3 liters for a biogenerator, 200 ml for a 250 ml spinner). Depending on the case, the culture medium used consists either of a mixture in equal parts of Nephros medium and of Williams medium to which is added a 40-kd PVP at a final concentration of 0.1%, or a mixture in equal parts of VPSFM medium and of Williams medium to which is added a 40-kd PVP at a final concentration of 0.1%, or a mixture in equal parts of VPSFM medium and of Williams medium to which is added donor calf serum at a final concentration of 4%, or, finally, a reference culture medium optimized for the industrial production of Vero cells, consisting of an ISCOVE base to which is added donor calf serum at a final concentration of 4%. The mechanical stirring of the medium is set at about 30 rpm. For the “low-density culture”, the surface aeration is performed by a mixture of air containing 5% CO2, while the oxygenation is performed deep down so as to have at the minimum an amount of dissolved oxygen in the medium equivalent to at least 10% of the maximum oxygen saturation of the medium. The culture may be maintained under these conditions for 5 to 7 days without changing or supplying medium either to the spinner or to the biogenerator. For the “high-density culture” produced in the biogenerator, the culture medium is renewed from the second day of culturing up to the seventh day of culturing at a rate of two volumes of culture medium per 24 hours (culturing according to a continuous culturing mode under perfusion). The mechanical stirring of the paddle is set at about 15 rpm. The amount of dissolved oxygen in the medium is equivalent to 25% of the maximum oxygen saturation of the medium.

1F—Study of the Metabolic Parameters of the Culturing

The monitoring of the essential nutrients and metabolites is performed by a Nova Biomedical analytical system (Bio Profile 200) measuring the pH, the osmolarity of the medium and the levels of glutamine, of glucose, of glutamate, of lactate, of ammonia and of certain ions such as Ca²⁺, K⁺ and Na⁺. This analyzer requires a 500 μl sample of medium supernatant.

The compounds that are the most advantageous to monitor are, on the one hand, the glucose and glutamine, that are sources of energy for the cell, and, on the other hand, their degradation products, namely lactate and ammonia. Measurement of these elements allows the cellular state of the culture to be controlled.

1G—Study of the Cell Growth

The cell growth is evaluated daily by means of the number of cells attached to the beads, by the technique of staining the nuclei according to the method of Sanford K. et al. (J. Nat. Cancer Inst., vol. 11, 773-795 (1951)) and of Van Wezel A. (Microcarrier culture of animal cells. Tissue culture: methods and applications, Kruse P. F. and Patterson M. K., Eds, Academic Press, New York, 372-377 (1973)).

5 ml of a homogeneous withdrawal of a culture from a biogenerator or spinner are introduced into a 15 ml tube. After separating out the beads by settling and removal of the supernatant, the beads colonized with the cells are rinsed twice with a phosphate buffer. 5 ml of a 0.1% solution of crystal violet are added to the final rinsing pallet. After incubating for 45 minutes at 37° C., the citric acid contained in the crystal violet was released and stained all the nuclei of the cells. The suspension of nuclei is then homogenized by stirring, and the nuclei are then counted on a Fuchs-Rosenthal slide. The number of nuclei reflects the number of cells attached to the microcarriers, which is related to the number of cells per ml of medium. The uncertainty of the measurement is ±10%. The results of the cell counts, expressed as a number of cells/ml, are given in the table below.

TABLE 1

| | N/W | N/W + PVP | VPSFM/W | VPSFM/W + PVP | VPSFM/W + SVD | ISCOVE + SVD |
|---|---|---|---|---|---|---|
| D0 | 675000* | 675000 | 405000 | 405000 | 405000 | 405000 |
| D1 | 307810 | 1562500 | 533000 | 781250 | 881000 | 573000 |
| D2 | 646875 | 2375000 | 997000 | ND | 2210000 | 1430000 |
| D3 | 868750 | 3250000 | 1390000 | 2006250 | 2230000 | 1740000 |
| D4 | 1765625 | 2906250 | 1670000 | 2162500 | 2590000 | 1950000 |
| D5 | 1187500 | 2468750 | 1800000 | 1993750 | ND | 2330000 |
| D6 | 1023440 | 2046875 | 1610000 | 1887500 | 1990000 | 1920000 |

Legends:

D0, D1, D2, D3, D4, D5 and D6 represent the withdrawal times for counting cells, the time D0 corresponding to the cell concentration in the medium after inoculating the cells in the Biogenerator in the case of a “low density” culture, D1 corresponding to the time after culturing for 24 hours, D2 corresponding to the time after 48 hours, D3 the time after 72 hours, D4 the time after 96 hours, D5 the time after 120 hours and D6 the time after 144 hours.

*: number of cells/ml

N/W: Nephros (Biowhittaker)/Williams medium

N/W+PVP: Nephros (Biowhittaker)/Williams medium supplemented with 0.1% of a 40-kd PVP

VPSFM/W: VPSFM (life technology)/Williams medium

VPSFM/W+PVP: VPSFM (life technology)/Williams medium supplemented with 0.1% of a 40-kd PVP

VPSFM+DCS: VPSFM/Williams medium supplemented with 4% donor calf serum

ISCOVE+DCS: Iscove medium supplemented with 4% donor calf serum

ND: not determined

Conclusions:

Although the operating conditions and the cell inoculation concentrations are identical, the N/W+PVP medium increases the proliferation of the Vero cells and allows significantly higher maximum cell concentrations to be reached than those obtained with the N/W medium alone (325 000 at D3 versus 1 765 625 at D4). The proliferation index found with the N/W+PVP medium is 1.84 and is very markedly higher than 1.2 (1+2×0.1 (10% being the measuring uncertainty specified in the experimental conditions). The same tendency is also observed with the VPSFM/W medium, in which the PVP also accelerates the cell growth (the proliferation index is 1.201).

The action of the PVP on the cell growth of the adherent cells is thus not dependent on the medium used. Furthermore, PVP 40 affords substantially similar results to those obtained with calf serum, since the cell concentrations measured during days 1 to 6 are substantially of the same order. The results obtained with a 360-kd PVP at a final concentration of 0.5% in the culture medium are similar to those obtained with a PVP 40. On the other hand, a medium containing a 10-kd PVP at a final concentration of 0.1% in the culture medium does not have a positive effect on the cell growth.

We also studied the growth of Vero cells in a biogenerator, placed under “high density” culture conditions and in which the culture medium is a mixture in equal parts of VPSFM and of Williams medium to which was added a 40-kd PVP at a final concentration of 0.1%. The values of the cell concentrations obtained between D0 and D6 are listed in Table 2 and expressed in millions of cells/ml.

TABLE 2

| | D0 | D1 | D2 | D3 | D4 | D5 | D6 |
|---|---|---|---|---|---|---|---|
| VPSFM/W + PVP | 2.05 | 2.00 | 2.85 | 4.05 | 5.85 | 5.95 | 5.80 |

These results show that the cell concentration increased threefold between D0 and D6. A medium free of serum of animal origin and containing a PVP as serum substitute can thus be used for “high density” culturing in a biogenerator.

### EXAMPLE 2

### Study of the Action of PVP on the Cell Death Rate

The operating conditions of the study are identical to those developed in paragraphs 1A to 1F.

The cell death rate is assessed daily by means of the number of dead cells or the measurement of the LDH (lactate dehydrogenase) found in the culture medium. The cell death rate is assessed by means of staining with trypan blue. Starting with a daily withdrawal of 1 ml of cell culture, 1 ml of 10% trypan blue is added. The dead cells which take the stain are counted using a Fuchs-Rosenthal cell. The measurement uncertainty is ±10%. The cell death rate results, expressed as the number of cells/ml, are given in Table 3.

The assessment of the cell death rate can also be determined by means of measuring the lactate dehydrogenase in the culture supernatant. Specifically, it has been shown that an assessment of the cell death rate during fermentation in bioreactors can be performed by measuring the LDH released into the culture supernatant. A daily withdrawal of 0.5 ml is centrifuged at 800 rpm for 5 minutes to remove the cells. It is then incubated with the mixture of reagents of the kit (Cytoxicity Detection kit (LDH), Boehringer Mannheim, Cat. No. 1644 793) according to the procedure described in the kit. An increase in the cell death rate or in the membrane lesions leads to an increase in the LDH activity, reflected by an increase in the optical density (or in the staining) in the test used. The assay is performed in 96-well flat-bottomed plates (Evergreen Scientific) with an untreated polystyrene lid. 200 μl of the supernatant to be assayed or of dilutions thereof are placed in two wells. 100 μl of reagent from the kit are added per well. After incubating for 10 minutes at 37° C. in a dry oven, the enzymatic reaction is quenched with 50 μl of 1N HCl. The optical density (OD) is measured at 490 nm using a microplate reader (Molecular devices Emax). The measuring precision is ±10%. The higher the OD, the higher the cell death rate. The comparative results between the various media tested, expressed as O.D. of the test sample, are collated in Table 4.

TABLE 3

| | | | VPSFM/W + | ISCOVE + |
|---|---|---|---|---|
| Withdrawal | N/W + PVP | VPSFM/W | PVP | FCS |
| D0 | N.D. | N.D. | N.D. | N.D. |
| D1 | < | 19063 | < | 51250 |
| D2 | < | 23125 | < | 48440 |
| D3 | < | 35313 | < | 52810 |
| D4 | < | 16875 | < | 42500 |
| D5 | < | 61560 | < | 75690 |

N.D.: not determined.
<: less than 5000 cells/ml

TABLE 4

| | | | VPSFM/W + | ISCOVE + |
|---|---|---|---|---|
| Withdrawal | N/W + PVP | VPSFM/W | PVP | FCS |
| D0 | N.D. | N.D. | N.D. | N.D. |
| D1 | 0.027 | 0.110 | 0.028 | 0.055 |
| D2 | 0.028 | 0.230 | N.D. | 0.073 |
| D3 | 0.065 | 0.390 | 0.066 | 0.153 |
| D4 | 0.112 | 0.510 | 0.115 | 0.155 |
| D5 | 0.140 | 0.600 | 0.226 | 0.268 |

N.D.: not determined

Conclusions:

Whereas the results obtained with media without serum or with serum show a cell death rate ranging between 10⁴ and 10⁵ dead cells/ml in the absence of PVP, those obtained with defined media supplemented with PVP show a very significant decrease in the cell death rate.

The results of the measurement of the LDH activity in the culture supernatants also show a lower activity in the culture supernatants obtained from media containing a PVP than those free of PVP. It may also be noted that the LDH activity is higher in the culture supernatants obtained from a serum-based medium than that present in media containing a PVP. These results thus correlate with those of the cell death rate. It may therefore be concluded that the culture media containing a PVP ensure better cell integrity by very substantially reducing the cell death rate, this action being greater than that observed with a medium containing calf serum.

### EXAMPLE 3

### Study of the Action of PVP on the Adhesion of Cells to Their Support

The operating conditions of the study are identical to those developed in paragraphs 1A to 1F.

To evaluate the effect of a PVP on the adhesion of cells, the number of cells which have become attached to the microcarriers 4 hours after inoculating the biogenerator or the Spinner according to the technique developed in paragraph 1G is determined. It is considered that, after 4 hours, the Vero cells are still in the same phase of their cell cycle, which means that the number of nuclei counted corresponds to the number of cells attached. The measurement uncertainty is ±10%. The results, expressed as the number of cells/ml, are given in the table below.

TABLE 5
With-

| drawal | N/W | N/W + PVP | VPSFM/W | VPSFM/W + PVP |
|---|---|---|---|---|
| 0 | 675000* | 675000 | 405000 | 405000 |
| 4 h | 453125 | 787500 | 394000 | 439250 |

Conclusions:

These results clearly show that the cell concentration is higher after 4 hours when the serum-free media contain a PVP.

### EXAMPLE 4

### Role of a PVP in the Growth, Adhesion and Mortality of Vero Cells Cultured in Rolling Flasks

The Vero cells as described in paragraph 1A are inoculated in 300 ml rolling flasks, (Ref.: Falcon 3007) at a rate of 30 000 to 50 000 cells per cm² of support. Various defined and nondefined culture media free of serum of animal origin but containing 0.1% of a 40-kd PVP were tested, including those described in paragraph 1A. A 100-kd PVP at 0.5% and also a 360-kd PVP at 0.05% were also tested using the same culture media.

The Vero cells in suspension in the various culture media tested are then introduced into rolling flasks which are then placed on rollers rotating at a speed of between 0.1 rpm and 0.5 rpm. Four hours after inoculating the flasks, and then daily for 6 days, a study of the growth and adhesion of the cells to the walls of the flask is performed on these rolling flasks, after having removed the culture medium, washed the flasks with a phosphate buffer and detached the cells with 2.5% trypsin (Sigma) diluted to 1/5000 in a citrate buffer. The cell counting is performed using trypan blue. The study of the cell death rate was also monitored over a period of 5 days starting from the measurement of the LDH in the culture supernatant. The results of these studies corroborate those described in the preceding examples and indicate that a PVP can be used in an average molecular weight range that is broad but nevertheless greater than 10 kd and in a broad range of concentration in a culture medium free of serum of animal origin to promote the proliferation and attachment of adherent cells and to very substantially reduce the cell death rate.

### EXAMPLE 5

### Role of a PVP in the Growth, Adhesion and Mortality of MRC5 Cells Cultured in Rolling Flasks

The MRC5 cells (Ref.: NIBSC (pdl8)) used between the 20^{th} and 50^{th} passage are inoculated in 300 ml rolling flasks (Ref.: Falcon 3007) at a rate of 50 000 cells per cm² of support. Various defined culture media free of serum of animal origin but containing 0.1% of a 40-kd PVP were tested, including those described in paragraph 1A. A 100-kd PVP at 0.1% and also a 360-kd PVP at 0.05% were also tested using the same culture media.

The rolling flasks containing the MRC5 cells and also the various culture media mentioned are then placed on rollers rotating at a speed of between 0.1 rpm and 0.5 rpm. Four hours after inoculating the flasks, and then daily for 4 days, a study of the growth and adhesion of the cells to the walls of the flask was performed on these rolling flasks after having removed the culture medium, washed the flasks with a phosphate buffer and detached the cells with 2.5% trypsin (Sigma) diluted to 1/5000 in a citrate buffer. The cell counting is performed using trypan blue. The study of the cell death rate was also monitored over a period of 4 days starting with the measurement of the LDH in the culture supernatant. The results of these studies correspond to those obtained with the Vero cells and show that the effects of a PVP on the cell growth, death rate and adhesion are not limited to a single cell type.

### EXAMPLE 6

### Influence of a PVP on the Production of Polio Viruses Obtained from Infected Vero Cells

6A—Cell Culturing

The Vero cells are cultured for 4 days on a plastic support in the Nephros/Williams defined culture medium containing or not containing a PVP with an average molecular weight of 40 kd, at a final concentration of 0.1% in the culture medium.

6B—Infection of the Cells and Viral Harvests

The culture medium is then removed, after which the cells are infected with a suspension of type 3 polio virus (sabin VERO 27/09/96 having a titer of 10⁹ DICC50/ml) prediluted in an infection medium so that the infection multiplicity is 0.1. The infection medium and viral propagation medium used are the Nephros (Biowhittaker)/Williams E medium supplemented or otherwise with a 40-kd PVP at a final concentration of 0.1%.

Withdrawals of culture supernatant are performed daily (to measure the viral activity of the polio virus on HEP2 cells) and are replaced with an equivalent amount of fresh medium identical to the viral propagation medium used. These harvests are continued up to the 7^{th} harvest (R1 to R7), the destruction of the cell carpet then being complete. The table below summarizes the results of the harvests carried out. The values of the viral titers obtained are expressed in log 10 of the viral infectious dose which induces in 50% of cases a cytotoxic effect on the HEP2 line (CCID).

TABLE 6
Table summarizing the titers of type 3 polio virus
according to the harvests and the culture media used
for the culturing of the Vero cells, the viral
infection and the viral propagation.

| Medium | R1 | R2 | R3 | R4 | R5 | R6 | R7 |
|---|---|---|---|---|---|---|---|
| N/W + PVP | 4.2 | 5 | 6.6 | 5.9 | 5 | 5 | 4.4 |
| N/W | 4 | 5 | 5 | 5.5 | 4.5 | 4.3 | 3.8 |

Conclusions

The addition of 0.1% 40-kd PVP to the Nephros/Williams chemically defined medium greatly increases the production of type 3 polio virus when compared with a PVP-free Nephros/Williams medium. The presence of a PVP in a culture medium free of serum of animal origin during the viral infection and/or propagation steps increases the production of viruses by the infected cells.

1. The use of a polyvinylpyrrolidone with an average molecular weight of between 20 kd and 360 kd in a culture medium free of serum of animal origin, to increase the proliferation of adherent nonrecombinant animal or human cells subjected to an agitation flow of the medium.

2. The use as claimed in claim 1, in which the proliferation index of the cells in this medium is greater than 1.5.

3. The use of a polyvinylpyrrolidone with an average molecular weight of between 20 kd and 360 kd in a culture medium free of serum of animal origin, to promote the adhesion of adherent animal or human cells subjected to an agitation flow of the medium.

4. The use of a polyvinylpyrrolidone with an average molecular weight of between 20 kd and 360 kd in a culture medium free of serum of animal origin, to reduce the death rate of adherent animal or human cells subjected to an agitation flow of the medium.

5. The use as claimed in any one of claims 1 to 4, in which the polyvinylpyrrolidone is noncrosslinked.

6. The use as claimed in any one of claims 1 to 5, in which the polyvinylpyrrolidone has an average molecular weight of 40 kd.

7. The use as claimed in any one of claims 1 to 6, in which the percentage of polyvinylpyrrolidone in the culture medium is between 0.01% and 2%.

8. The use as claimed in claim 7, in which the percentage of polyvinylpyrrolidone in the culture medium is 0.1%.

9. The use as claimed in any one of claims 1 to 8, in which the culture medium has a chemically defined composition.

10. The use as claimed in one of claims 1 to 9, for the production of nonrecombinant adherent human or animal cells in batch or feed batch mode or in continuous culture mode.

11. A process for producing adherent nonrecombinant animal or human cells, comprising:
(i) a first step in which a suspension of cells in a serum-free culture medium containing a polyvinylpyrrolidone with an average molecular weight of between 20 kd and 360 kd is inoculated in a culturing device comprising an adhesion support;
(ii) a second step in which the cells multiply under agitation in the same culture medium;
(iii) a third step in which the cells are harvested when they have reached a growth stage.

12. The process as claimed in claim 11, in which the polyvinylpyrrolidone has an average molecular weight of 40 kd.

13. The process as claimed in claim 11 or 12, in which the percentage of polyvinylpyrrolidone in the culture medium is between 0.01% and 2%.

14. The process as claimed in one of claims 11 to 13, in which the percentage of polyvinylpyrrolidone in the culture medium is 0.1%.

15. The process as claimed in one of claims 11 to 14, in which the adhesion support consists of microcarriers.

16. The process as claimed in claim 15, in which the microcarriers consist of a dextran matrix (cytodex 1).

17. The process as claimed in one of claims 11 to 16, in which the culturing device is a biogenerator.

18. The process as claimed in one of claims 11 to 17, in which the culture mode is the batch or feed batch mode or the continuous culture mode.

19. Process as claimed in one of claims 11 to 18, in which the cells are cells of the Vero or MRC5 line.

20. A process for producing viruses, according to which:
(i) cells are infected with viruses in a first culture medium;
(ii) the viruses are left to propagate and are harvested in a second culture medium;
at least one of the two media being a medium free of serum of animal origin and containing a polyvinylpyrrolidone with an average molecular weight of beween 20 kd and 360 kd.

21. The process as claimed in claim 20, in which the first culture medium is free of serum of animal origin and contains a polyvinylpyrrolidone with an average molecular weight of between 20 kd and 360 kd.

22. The process as claimed in claim 20 or 21, in which the second culture medium is free of serum of animal origin and contains a polyvinylpyrrolidone with an average molecular weight of between 20 kd and 360 kd.

23. The process as claimed in one of claims 20 to 22, in which the viruses are produced from cells of the Vero or MRC5 line.

24. The process as claimed in claim 23, in which the viruses produced are polio virus, rabies virus, hepatitis A virus or rubella virus.
